(19) United States
(12) Patent Application Publication
(10) Pub. No.: US 2020/0221697 A1
(43) Pub. Date: Jul. 16, 2020

(54) Modified Polyvinylchloride Surface with Antibacterial and Antifouling Functions

(71) Applicant: The Trustees of Indiana University, Indianaplis, IN (US)

(72) Inventor: Dong Xie, Carmel, IN (US)

(73) Assignee: The Trustees of Indiana University, Indianapolis, IN (US)

(21) Appl. No.: 16/739,180

(22) Filed: Jan. 10, 2020

Related U.S. Application Data
(60) Provisional application No. 62/791,056, filed on Jan. 11, 2019.

(51) Int. Cl. A01N 43/36; C08F 226/10; C08F 224/00; A61L 31/04

(52) U.S. Cl. CPC A01N 43/36; C08F 226/10; A61L 2300/404; A61L 31/048; C08F 224/00

(57) ABSTRACT

Disclosed are materials having an antifouling and a biocidal property. The materials include a polyvinylchloride plastic covalently linked to a polymer, where the polymer includes an antifouling component and a biocidal component.

### CROSS-REFERENCE TO RELATED APPLICATIONS

This application is related to and claims priority to U.S. Provisional patent application, Ser. No. 62/791,056, entitled “A modified polyvinylchloride surface with antibacterial and antifouling functions” filed on Jan. 11, 2019, the disclosure of which is hereby incorporated by reference in its entirety.

Surface modification is crucial to a variety of biomaterials applications. Some surfaces need to be modified to be cell or tissue-integrated whereas others require modification to be antifouling, i.e., lower or no cell adhesion. In light of biomedical applications, two main factors limit use of polymeric materials in surface-related medical devices: their highly hydrophobic surface while being used in contact with body fluid and/or blood and bacterial infection or contamination while bacteria attach and/or grow on surface. A hydrophobic surface can cause cell adhesion, bacterial adhesion and nonspecific protein adsorption. Cell adhesion and protein adsorption can lead to blood flow blockage if polymers are used internally. Bacterial attachment and biofilm formation can cause biomaterials-relevant infections. Attempts have been made to achieve polymer surface modifications for reduced cell adhesion and protein adsorption. For example, entire hydrophilic or amphiphilic polymers have been used to manufacture medical devices. Modifying a surface of the formed medical devices have been attempted.

Polyvinylchloride is a commonly used thermoplastic polymer for biomedical application, due to its low cost, easy processing and low toxicity. This polymer has been used in making many cardiovascular devices such as catheters, blood vessels, artificial heart pumps, and dialysis devices. However, like most other polymers, polyvinylchloride is very hydrophobic, which leads to cell adhesion and protein adsorption, if it contacts body fluid or blood, and bacteria contamination if it is not sterile.

In terms of preventing bacterial infection, three strategies are used to develop antimicrobial surfaces. One is to generate a non-fouling surface which can reduce or resist cell and bacterial attachment. In other words, surface is made to be very hydrophilic. The other is to incorporate leachable antibacterial compounds such as zinc ion, silver ion, chlorhexidine, iodine or antibiotics into medical devices. Slow release of the biocides kills or inhibits many microorganisms. However, these strategies suffer from a number of shortcomings which include short-term effectiveness but long-term run-out, potential cytotoxicity to surrounding tissues, and potential development of microbial antibiotic resistance caused by the gradually decreasing concentrations of the released compounds. Another approach is to create antimicrobial surfaces by chemically linking antibacterial compounds onto the surfaces, which allows the attached compounds to kill or inhibit bacteria by simple contact. This strategy is thought to be unique in preventing long-term disinfection and reducing the risk for formation of antibiotic-resistant bacteria. This is believed to one of the most effective strategies. Due to the fact that quaternary ammonium salts can be simply derivatized and easily incorporated into a polymer, their derivatives have been widely and extensively studied for contact-mediated microbial inhibition. However, it was reported that interactions between quaternary ammonium salts and proteins can reduce antimicrobial effectiveness.

It was found that the derivatized 2(5H)-furanone compounds exhibited significant antibacterial functions without proteins interference. This antibacterial effect has been validated on dental restoratives. These derivatives were covalently linked to dental polymers or dental composites, resulting in killing bacteria or inhibiting bacterial growth by simple contact but not via release or leaching. This greatly reduces the potential cytotoxicity from the antibacterial derivatives to the surrounding tissues. It was also found that the modified restoratives did not significantly interact with human saliva, limiting negative protein effects on antibacterial functions, unlike quaternary ammonium salt-containing materials.

In this invention, a new polymer composed of both antifouling moieties and antibacterial residues is coated onto a polyvinylchloride surface via an effective surface coating technique and completing the coating process in a mild condition to create an antibacterial and antifouling surface.

Surfaces with antibacterial and hydrophilic properties are very attractive to cardiovascular applications. In this invention, a novel antibacterial and hydrophilic polymer was synthesized and immobilized onto a surface of polyvinylchloride via an effective and mild surface coating technique. The surface coated with a terpolymer constructed with N-vinylpyrrolidone, 3,4-Dichloro-5-hydroxy-2(5H)-furanone derivative and succinimide residue was evaluated with cell adhesion, bacterial adhesion and bacterial viability. 3T3 mouse fibroblast cells and two bacteria species were used to evaluate surface adhesion and antibacterial activity. Results showed that the polymer-modified polyvinylchloride surface exhibited not only significantly decreased 3T3 fibroblast cell adhesion with a 66-87% reduction but also significantly decreased bacterial adhesion with 69-87% and 52-74% reduction of Pseudomonas aeruginosa and Staphylococcus aureus attachment, respectively, as compared to original polyvinylchloride. Furthermore, the modified polyvinylchloride surfaces exhibited significant antibacterial functions by inhibiting bacterial growth (75-84% and 78-94% inhibition of Pseudomonas aeruginosa and Staphylococcus aura's; respectively, as compared to original polyvinylchloride) and killing bacteria. These results demonstrate that covalent polymer attachment conferred antifouling and antibacterial properties to the polyvinylchloride surface.

### BRIEF DESCRIPTION OF THE DRAWINGS

The patent or application file contains at least one drawing executed in color. Copies of this patent or paten application publication with color drawing(s) will be provided by the Office upon request and payment of the necessary fee.

FIGS. 1A-1B show the scheme for (A) PVDCS synthesis and (B) surface coating with PVDCS.

FIGS. 2A-2D show FT-IR spectra for PVDCS synthesis for: (A) SA; (B) ACDF; (C) NVP; and (D) PVDCS.

FIGS. 3A-3E show FT-IR spectra for PVC surface coating as follows: (A) PVC (B) PVCN3; (C) PVCPA; (D) PVCNCO; and (E) PVCPEI.

FIG. 4 shows 3T3 mouse fibroblast adhesion on PVC and surface-modified PVC with different polymer coatings.

FIG. 5 shows bacteria adhesion on PVC and surface-modified PVC with different polymer coatings for P. aeruginosa and S. aureus.

FIG. 6 shows bacterial viability after incubating with PVC and its surface-modified PVC with different polymer coatings for P. aeruginosa and S. aureus.

FIGS. 7A-7H show images of S. aureus after incubating with PVC and its surface-modified PVC disks: (A) PVC, (B) PVCN3, (C) PVCPA, (D) PVCPEI, (E) PVDCS8758, (F) PVDCS82108, (G) PVDCS77158, and (H) PVDCS72208.

One embodiment of the invention is a material having an antifouling and a biocidal property. The material includes a polyvinylchloride plastic covalently linked to a polymer, where the polymer includes an antifouling component and a biocidal component. The polymer may also include a coupling component. The coupling component may be N-succinicmidyl acrylate. The antifouling component of the polymer may be N-vinylpyrrolidone. The biocidal component of the polymer may exert an antibacterial effect, and the antibacterial effect may be exerted against a bacterium selected from the group consisting of P. aeruginosa and S. aureus. The biocidal component of the polymer may include 5-acryloylethyleneglycol-3-4-dichloro-2(5H)-furanone. The molar ratio of the antifouling agent to the biocidal component may be from about 87:5 to about 72:20. The molar ratio of the antifouling component to the biocidal component to the coupling component may be from about 87:5:8 to about 72:20:8.

A further embodiment of the invention is a medical device that includes a surface material, where the surface material has an antifouling and a biocidal property. The surface material includes a polyvinylchloride plastic covalently linked to a polymer, where the polymer includes an antifouling component and a biocidal component. The polymer may further include a coupling component. The coupling component may be N-succinicmidyl acrylate. The antifouling component of the polymer may be N-vinylpyrrolidone. The biocidal component of the polymer exerts an antibacterial effect. The biocidal component of the polymer may exert an antibacterial effect against a bacterium selected from the group consisting of P. aeruginosa and S. aureus. The biocidal component of the polymer may include 5-acryloylethyleneglycol-3-4-dichloro-2(5H)-furanone. The molar ratio of the antifouling agent to the biocidal component may be from about 87:5 to about 72:20. The molar ratio of the antifouling component to the biocidal component to the coupling component may be from about 87:5:8 to about 72:20:8.

Yet another embodiment of the invention is a polymer having the structure:

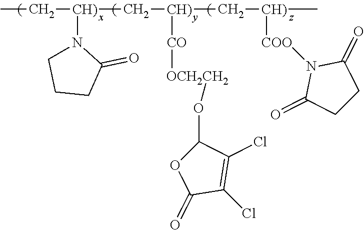

where x, y and z are integers between 1 and 10,000. In the polymer, z may equal 8. The polymer may be covalently linked to polyvinylchloride.

Acryloyl chloride, N-hydroxysuccinimide, triethylamine, 4-methoxyphenol, 2-hydroxyethyl acrylate, 3,4-dichloro-5-hydroxy-2(5H)-furanone, p-toluenesulfonic acid, toluene, 4-methoxyphenol, sodium azide, tetrabutylammonium bromide, 1,6-diisocyanatohexane, propargyl alcohol, dibutyltin dilaurate, 2,2′-azobisisobutyronitrile, N-vinylpyrrolidone (NVP), poly(ethyleneimine) (PEI), tetrahydrofuran, dimethylformamide, diethyl ether, copper sulfate, sodium ascorbate, sodium chloride, anhydrous magnesium sulfate and sodium bicarbonate were used as received from Sigma-Aldrich Co. (Milwaukee, Wis.) without further purifications. Polyvinylchloride (PVC) sheet (0.5 mm thick) was received from Interstate Plastics (Sacramento, Calif.).

Synthesis of functional antibacterial hydrophilic polymer was carried out in three steps, i.e., synthesis of N-succinimidyl acrylate (SA), synthesis of 5-acryloylethyleneglycol-3,4-dichloro-2(5H)-furanone (ADCF) and synthesis of poly(NVP-ADCF-SA) or PVDCS.

SA synthesis: Acryloyl chloride (0.1 mol) was slowly added to a solution containing N-hydroxysuccinimide (0.1 mol), triethylamine (0.1 mol), 4-methoxyphenol (0.1 mol % of triethylamine) and tetrahydrofuran. The reaction was conducted at 23° C. for 24 h and the by-product triethylamine-hydrogen chloride was filtered. The product, a white solid, was recovered after removing tetrahydrofuran with a rotary evaporator and drying in vacuo.

ADCF synthesis: A mixture of 3,4-dichloro-5-hydroxy-2(5H)-furanone (0.1 mol), 2-hydroxyethyl acrylate (0.12 mol), 4-methoxyphenol (0.1 mol %), toluene and p-toluenesulfonic acid (2 mol %) was refluxed at 100-110° C. for 3-4 h. After toluene was removed via the rotary evaporator, the recovered crude product ADCF was dissolved in diethyl ether, washed with saturated sodium bicarbonate solution, brine and distilled water, and dried with anhydrous magnesium sulfate, followed by removing solvent by the rotary evaporator.

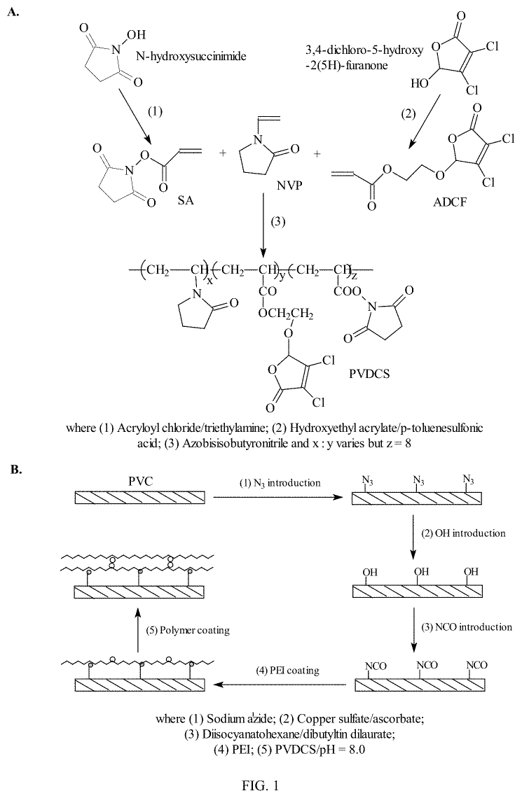

PVDCS synthesis: 2,2′-azobisisobutyronitrile (1% by mole) was added to a solution containing N-vinylpyrrolidone, ADCF and SA at a molar ratio of 87/2/8, 82/10/8, 77/15/8 or 72/20/8 in N,N′-dimethylformamide. After the reaction was carried out under a N₂ blanket at 64° C. for 24 h, the PVDCS polymer was purified with diethyl ether and dried in vacuo. The scheme for synthesis is shown in FIG. 1A.

Polyvinylchloride (PVC) sheet was cut into 7-mm diameter disks. Then disks were placed in a container with sodium azide (20%, w/v), tetrabutylammonium bromide (2% w/v) and 10 ml distilled water with stirring. After running the reaction at 80° C. for 7 h, the disks were washed three times with distilled water (formation of PVC with azido groups: PVCN3), followed by placing them in a container with propargyl alcohol (16%), copper sulfate (2%), tetrabutylammonium bromide (1%), sodium ascorbate (0.5%) and distilled water (15 ml). The reaction was conducted at 50° C. for 3 h and then the disks were washed three times with distilled water, resulting in the disks having hydroxyl groups on the surfaces (formation of PVC with hydroxyl groups: PVCPA). The modified PVC disks were then placed in a container with 1,6-diisocyanatohexane (20%), dibutyltin dilaurate (1%) and hexane (10 ml) with stirring. After running the reaction at 40° C. for 1.5 h, the disks were washed three times with hexane (formation of PVC with isocyanate groups: PVCNCO), followed by placing them in a container with 5% PEI solution. After coating at 23° C. overnight, the disks were washed three times with distilled water (formation of PVC coated with PEI having amino groups on the surface: PVCPEI) and then dried in an oven. Finally the antibacterial and hydrophilic PVDCS polymer was coated onto the PVCPEI surface. Briefly, 10% (wt/wt) of the synthesized PVDCS in distilled water was added to a solution containing buffer (pH=8.5) and acetone (1:1 v/v). Then the amine-modified PVC disks were added upon dissolution of the polymer. The reaction was conducted at 24° C. for 30 min, followed by washing the modified disks three times with distilled water before evaluation. The scheme for modification is shown in FIG. 1B.

The synthesized polymer and surface-modified disks were characterized and evaluated with Fourier transform-infrared (FT-IR) spectroscopy. The surface functional groups of the modified PVC were characterized with attenuated total reflectance FT-IR. FT-IR spectra were acquired on a FT-IR spectrometer (Mattson Research Series FT/IR1000, Madison, Wis.).

NIH-3T3 mouse fibroblasts were cultured in high glucose Dulbecco's Modified Eagle Medium (DMEM, Lonza) supplemented with 10% fetal bovine serum (FBS, Invitrogen), 5 mg/ml penicillin and 5 mg/ml streptomycin (Invitrogen Inc., Singapore). After maintaining at 37° C. under a humidified atmosphere of 5% CO₂ for 24 h, the cells were harvested from the culture flask by the addition of a 5.3 mM trypsin-EDTA (ThermoFisher Scientific) solution in PBS and centrifuged at 1200 rpm for 3 min, followed by removing trypsin and re-suspending the cell pellets in DMEM medium supplemented with 10% FBS to a density of 5×10⁴ cells/mL. The formed cell suspension (1 mL) was then added to each well containing the disk specimen in a 24-well plate and cultured for 48 h, before the disk was washed with PBS to remove non-adherent cells. The cells attached to the disk were harvested by the addition of trypsin, followed by counting and imaging with an inverted microscope (Nikon Ti-E, Melville, N.Y.). Triplicate samples were used to obtain a mean value for each material.

The bacterial adhesion test was conducted following slightly modified published protocols as follows. Colonies of bacteria were suspended in 5 mL of tryptic soy broth, supplemented with 1% sucrose, to form a suspension with 10⁸ CFU/mL of bacteria and cultured for 24 h. P. aeruginosa, S. aureus and E. coli were assessed. After washing with 70% ethanol for 10 s and sterile water three times, the disk specimen was incubated with bacteria in tryptic soy broth at 37° C. for 24 h under 5% CO₂. Then the disk was rinsed with sterile PBS and de-ionized water to remove non-adherent bacteria. The adhered bacteria were eluted from the surfaces by ultrasonic treatment in 1 ml sterile PBS for 10 min. Bacterial CFU was enumerated by agar plate counts. Data represent a mean value for each material based on triplicate samples.

The bacterial viability test was carried out by suspending bacterial colonies in 5 mL of tryptic soy broth, supplemented with 1% sucrose, to form a suspension with 10⁸ CFU/mL of bacteria and incubated for 24 h. Both P. aeruginosa and S. aureus were assessed. The disk specimen was sterilized with 70% ethanol for 10 s and incubated with the bacterial suspension in tryptic soy broth at 37° C. for 48 h under 5% CO₂. To 1 mL of the above bacterial suspension, 3 μL of a green/red (1:1 v/v) dye mixture (LIVE/DEAD BacLight bacterial viability kit L7007, Molecular Probes, Inc., Eugene, Oreg., USA) was added, followed by vortexing for 10 s, sonicating for 10 s, vortexing for another 10 s and keeping in dark for about 15 min before analysis. Then, 20 μL of the stained bacterial suspension was added onto a glass slide and viable bacteria (green) were imaged with an inverted fluorescence microscope (EVOS FL, AMG, Mill Creek, Wash., USA). A bacteria suspension without disks was used as control and viable bacteria counts from the suspension were used as 100%. Viability was analyzed by counting from the recorded images. Triplicate samples were used to obtain a mean value for each material.

One-way analysis of variance (ANOVA) with the post hoc Tukey-Kramer multiple-range test was used to determine significant differences of each measured property or activity among the materials in each group. A level of α=0.05 was used for statistical significance.

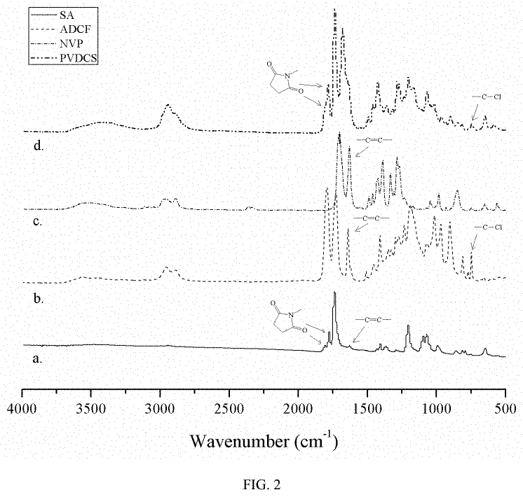

FIGS. 2A-2D show a set of FT-IR spectra for SA (A), ADCF (B), NVP (C) and PVDCS (D). In comparison with all the four spectra, the peaks around 1620-1655 for C=C group disappear in spectrum 2D, which corresponds to those at 1652 and 1629 for SA from spectrum 2A, 1639 for ADCF from spectrum 2B as well as 1629 for NVP from spectrum 2C. A broader and stronger peak at 3200 for amide group appears in spectrum 2D, which corresponds to that for NVP from spectrum 2C. Two small peaks at 1805 and 1778 for succinimidyl group (amide I) appear in spectrum 2D, which corresponds to the peaks at 1805 and 1776 for SA from spectrum 2A. A small peak at 750 for C-Cl group appears in spectrum 2D, which corresponds to that for ADCF from spectrum 2B. These changes confirmed the PVDCS formation.

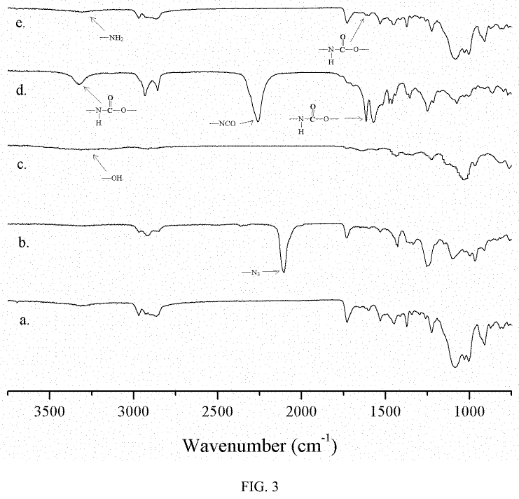

FIG. 3A-3E shows a set of FT-IR spectra for PVC (A), PVCN3 (B), PVCPA (C), PVCNCO (D) and PVCPEI (E). In comparison with spectra a and b, the appearance of a strong new peak at 2104 for azido group confirmed that azido groups were successfully attached onto the PVC surface by replacing some chlorine groups. By comparing spectra b and c, the azido peak disappeared and a broad new peak appeared between 3000 and 3700, indicating the hydroxyl group formation on the PVC surface. In comparison with spectra 3C and 3D, the appearance of new peaks at 3340 and 1650 for urethane group and at 2261 for isocyanate group confirmed that isocyanate groups were successfully attached onto the PVC surface by the reaction between hydroxyl and isocyanate groups. In comparison with 3D and 3E, appearance of a broad peak at 3400 and disappearance of isocyanate group at 2261 confirmed the successful coating of PEI on the PVC surface.

The medical devices used in cardiovascular applications require minimum microbial adhesion and low cell attachment. To achieve this, the surface was coated by using a newly prepared polymer containing both hydrophilic and antibacterial moieties, which not only can prevent mammalian cell adhesion but also reduce or prevent bacteria from infection. A simple and effective coupling technique was applied that has been broadly applied for protein coupling, i.e., coupling carboxyl with primary amino groups in water at pH=8.0 with N-hydroxysuccinimide.

Medical device-associated microbial infections are a significant problem associated with device implantation. These infections are associated with almost each type of medical device. Affected medical devices include, but are not limited to, catheters, vascular grafts and ureteral stents. Killing or inhibiting bacteria by touch or simple contact has attracted special attention recently. Quaternary ammonium salts and their derivatives, due to their potent antimicrobial functions, are used for a number of biomedical and pharmaceutical applications. These materials have shown capability of inhibiting and/or killing those bacteria that demonstrate resistance to cationic antibacterial compounds. However, these potent compounds have also shown some weakness while interacting with proteins such as human saliva. For example, oral saliva can significantly and negatively affect the antibacterial activity of these compounds. This undesirable result has been attributed to electrostatic interactions between these quaternary ammonium salts and proteins in saliva.

Furanone-containing antimicrobial compounds have been reported to show a broad spectrum of biological and physiological properties including but not limited to antibiotic, antitumor, haemorrhagic and insecticidal activities. 3,4-dichloro-5-hydroxy-2(5H)-furanone-containing polymer-composed dental composites have been found effective in inhibiting the growth of the oral bacterium Streptococcus mutans. The present invention introduces 3,4-dichloro-5-hydroxy-2(5H)-furanone through a polymerizable molecule 2-hydroxyethyl methacrylate via a covalent bond linkage into the hydrophilic PVDCS, covalently link the PVDCS to the activated PVC surface. The attached polymer imparts significant antifouling and antibacterial properties to the modified surface.

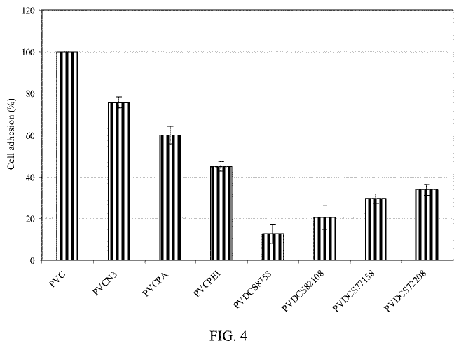

FIG. 4 shows the effect of the PVDCS polymers on cell surface adhesion by 3T3 mouse fibroblasts. The cell adhesion was in the decreasing order of PVC>PVCN3>PVCPA>PEI>PVDCS72208>PVDCS77158>PVDCS82108>PVDCS8758 (p<0.05). A hydrophobic surface has higher affinity to proteins, cells and even bacteria. PVC is a very hydrophobic, biofouling material. The modified PVCN3, PVCPA and PVCPEI showed significantly reduced cell adhesion (24%, 40% and 55% reduction, respectively, compared to original PVC), probably due to significantly decreased hydrophobicity. Azido group is known for its polarity. Both hydroxyl groups on PVCPA and amino groups on PVCPEI are hydrophilic. The surfaces modified with the antibacterial and hydrophilic polymers exhibit a further significant decrease in adhesion: PVDCS72208, PVDCS77158, PVDCS82108 and PVDCS8758 exhibited 66%, 70%, 80% and 87% cell adhesion reduction, respectively. The individual components of PVDCS each possess qualities contributing to overall functionality. NVP is very hydrophilic monomer and its formed polymers are used as blood substitutes due to their excellent blood-compatibility. ADCF exhibits antimicrobial and antitumor properties. SA has been used for coupling amino groups with carboxyl groups in protein chemistry. PVDCS8758 represents a molar ratio of 87/5/8 for NVP/ADCF/SA, which contains the highest ratio of NVP (hydrophilic component) and lowest ratio of ACDF (antibacterial component) whereas PVDCS72208 contains the lowest hydrophilic component but the highest antibacterial component. The more NVP on the surface, the lower the surface adhesion of the 3T3 cells.

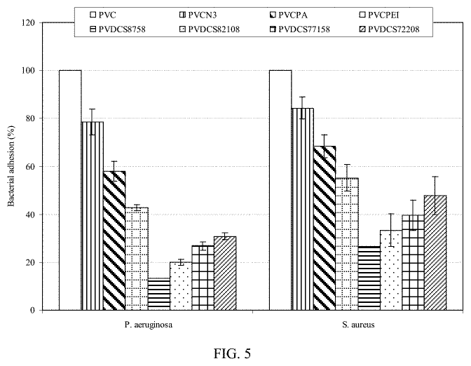

FIG. 5 shows the effect of the PVDCS polymers on surface bacteria adhesion. Bacteria adhesion exhibited a pattern similar to that of 3T3 fibroblast adhesion, as shown in FIG. 4. After 24 h incubation with bacteria, PVC and its modified surfaces were evaluated, considering adhesion to PVC as 100%. We found that bacteria attached to the disks in the following decreasing order: PVC>PVCN3>PVCPA>PVCPEI>PVDCS72208>PVDCS77158>PVDCS82108>PVDCS8758. The modified surfaces showed a significant bacterial adhesion reduction of 21%, 42%, 57%, 87%, 80%, 73% and 69% with P. aeruginosa and 16%, 32%, 45%, 74%, 67%, 60% and 52% with S. aureus for PVCN3, PVCPA, PVCPEI, PVDCS8758, PVDCS82108, PVDCS77158 and PVDCS77208, respectively, as compared to original PVC. In addition, S. aureus showed higher adhesion than P. aeruginosa. Again, PVC is a highly hydrophobic polymer. It showed the highest bacteria adhesion. The azido-modified PVC showed reduced bacterial adhesion. Note that the azido group is more hydrophilic than PVC. After the azido group was converted to hydroxyl group and then amino group, the bacteria adhesion was further reduced due to hydrophilic nature of both hydroxyl and amino groups. The PVDCS-modified PVC displayed further reduced bacterial adhesion. Similar to the results shown in FIG. 4, the PVDCS8758 showed the lowest bacterial adhesion but the one with highest ADCF showed the highest bacterial adhesion, although the adhesion values were still significantly lower than for PVC, PVCN3, PVCPA and PVCPEI.

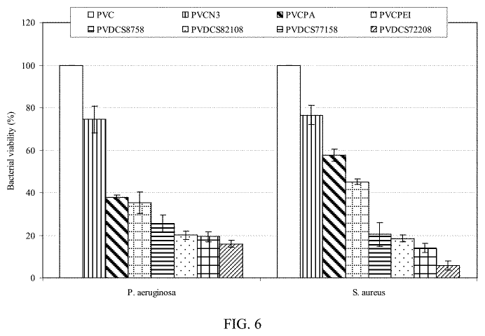

FIG. 6 shows the effect of the PVDCS polymers on viability of two bacterial species in the supernatant above the disks. Bacterial viability in the presence of the disk was found in the following decreasing order: PVC>PVCN3>PVCPA>PVCPEI>PVDCS8758>PVDCS82108>PVDCS77158>PVDCS72208. S. aureus showed lower viability than P. aeruginosa. Although PVCN3, PVCPA and PVCPEI did not contain any antibacterial residues, they still showed significantly decreased P. aeruginosa viability with reduction of 24%, 62% and 65% for PVCN3, PVCPA and PVCPEI and S. aureus viability with reduction of 23%, 42% and 55% for PVCN3, PVCPA and PVCPEI, as compared to original PVC. The result suggests that PVCN3, PVCPA and PVCPEI have a bacterial inhibition capability. PVCN3 has shown bacterial inhibition activity. The amine-containing polymers such as polyimine and polylysine has been shown to have antibacterial function. The antibacterial activity exhibited by PVCPA can be attributed to the triazole moieties produced from the reaction between acetylene groups from propargyl alcohol and azido groups on PVCN3. The triazole moieties have been shown to have an antimicrobial activity. By comparing with PVCN3, PVCPA and PVCPEI, the surfaces modified with antibacterial and hydrophilic polymers exhibited a dramatic viability reduction. P. aeruginosa and S. aureus displayed reduction values of 75% and 80% for PVDCS8758, 80% and 78% for PVDCS82108, 81% and 86% for PVDCS77158, and 84% and 94% for PVDCS72208, respectively, as compared to original PVC. The result is plausible because the more antibacterial component on the polymer or on the PVC surface, the lower the viability or higher bacterial inhibition is observed. These results demonstrate that the inventive polymer-coated surfaces can kill bacteria by contact.

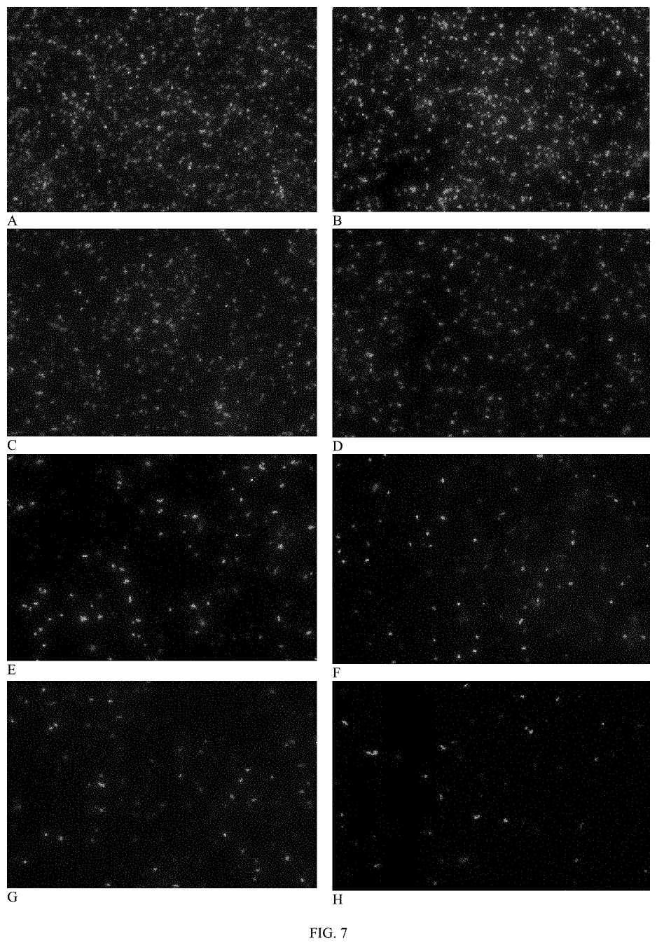

FIGS. 7A-7H show a set of photo-images of S. aureus viability after incubating with original PVC and modified PVC disks. The images depicted in FIGS. 7A-7Ha represent (A) PVC, (B) PVC-N₃, (C) PVCPA, (D) PVCPEI, (E) PVDCS8758, (F) PVDCS82108, (G) PVDCS77158, and (H) PVDBS72208. PVC showed the highest numbers of bacteria (green dots), followed by PVC-N₃, PVCPA, PVCPEI, PVDCS8758, PVDCS82108, PVDCS77158 and PVDCS72208. Nearly no red bacteria (dead cells) were observed from FIGS. 7A-7D for PVC, PVCN3, PVCPA and PVCPEI. However, red bacteria (dead cells) are observed from FIGS. 7E-7H. The images of PVDCS72208 showed only a few living bacteria cells (green) but more dead cells (red). Because PVC-N3, PVCPA and PVCPEI did not contain any antibacterial substances on the surfaces, they only inhibited bacterial growth but did not actively kill bacteria. With the antibacterial and hydrophilic polymer-coated PVC, however, not only bacteria growth were inhibited but also bacteria were actively killed, which led to significantly reduced living bacteria numbers and increased dead bacteria. Furthermore, increasing antibacterial component ADCF on polymers further decreased the living bacteria and increased the dead bacteria.

The inventive PVDCS polymer-coated PVC surfaces demonstrated an attractive antifouling property with significantly decreased mammalian cell and bacterial adhesion. Meanwhile, the polymer-coated surfaces also exhibited the capability of not only inhibiting bacterial growth but also killing bacteria, which would enhance antimicrobial activity of PVC and may also reduce the risk to bacterial infection due to insufficient sterilization.

A novel antifouling and antibacterial polymer was synthesized and immobilized the polymer onto hydrophobic surface of polyvinylchloride. The modified surface not only exhibited significantly reduced cell adhesion with a 66-87% decrease to 3T3 fibroblast but also showed significantly decreased bacterial attachment with 69-87% and 52-74% decrease to P. aeruginosa and S. aureus, respectively, as compared to original PVC. Furthermore, the polymer-modified PVC surface demonstrated significant antibacterial functions by inhibiting bacteria growth with reduction of 75-84% to P. aeruginosa and 78-94% to S. aureus, as compared to original PVC and killing bacteria as well. This invention has the ability to prevent medical device-related infections or complications.

Various modifications and additions can be made to the embodiments disclosed herein without departing from the scope of the disclosure. For example, while the embodiments described above refer to particular features, the scope of this disclosure also includes embodiments having different combinations of features and embodiments that do not include all of the described features. Thus, the scope of the present disclosure is intended to embrace all such alternatives, modifications, and variations as fall within the scope of the claims, together with all equivalents.

All publications, patents and patent applications referenced herein are hereby incorporated by reference in their entirety for all purposes as if each such publication, patent or patent application had been individually indicated to be incorporated by reference.

### REFERENCES

Ratner B D, Hoffman A S, Schoen F J, Lemons J E. Biomaterials Science: An Introduction to Materials in Medicine. 3rd edn. San Diego, Calif.: Elsevier Academic Press; 2012.
Shedden L, Oldroyd K, Connolly P. Current issues in coronary stent technology. Proc. Inst. Mech. Eng. H. 2009; 223: 515-524.
Donelli G. Vascular catheter-related infection and sepsis. Surg. Infect. 2006; 7: S25-27.
Cheng X, Canavan H E, Graham D J, Castner D G, Ratner B D. Temperature dependent activity and structure of adsorbed proteins on plasma polymerized N-isopropyl acrylamide. Biointerphases 2006; 1: 61-72.
Huber D L, Manginell R P, Samara M A, Kim B I, Bunker B C. Programmed adsorption and release of proteins in a microfluidic device. Science 2003; 301: 352-354.
Yu Q, Zhang Y, Chen H, Wu Z, Huang H, Cheng C. Protein adsorption on poly(N-isopropylacrylamide)-modified silicon surfaces: effects of grafted layer thickness and protein size. Colloids Surf. B. 2010; 76: 468-474.
Chen L, Liu M, Bay H, Chen P, Xia F, Han D, Jiang L. Antiplatelet and thermally responsive poly(N-isopropylacrylamide) surface with nanoscale topography. J. Am. Chem. Soc. 2009; 131: 10467-10472.
Uchida K, Sakai K, Kwon O H, Ito E, Aoyagi T, Kikuchi A, Yamato M, Okano T. Temperature-sensitive poly(N-isopropylacrylamide)-grafted surfaces modulate blood platelet interactions. Macromol. Rapid Commun. 2000; 21: 169-73.
Alarcon C D H, Farhan T, Osborne V L, Huck W T S, Alexander C. Bioadhesion at micro-patterned stimuli-responsive polymer brushes. J. Mater. Chem. 2005; 15: 2089-2094.
Cunliffe D, Alarcon C D, Peters V, Smith J R, Alexander C. Thermoresponsive surface-grafted poly(N-isopropylacrylamide) copolymers: effect of phase transitions on protein and bacterial attachment. Langmuir 2003; 19: 2888-2899.
Tan D, Li Z, Yao X, Xiang C, Tan H, Fu Q. The influence of fluorocarbon chain and phosphorylcholine on the improvement of hemocompatibility: a comparative study in polyurethanes. J. Mater. Chem. B. 2014; 2: 1344-1353.
You I, Kang S M, Byun Y, Lee H. Enhancement of blood compatibility of poly(urethane) substrates by mussel-inspired adhesive heparin coating. Bioconjug. Chem. 2011; 22: 1264-1269.
Garrett T R, Bhakoo M, Zhang Z. Bacterial adhesion and biofilms on surfaces. Prog. Nat. Sci. 2008; 18: 1049-1056.
Ren Z, Chen G, Wei Z, Sang L, Qi M. Hemocompatibility evaluation of polyurethane film with surface-grafted poly(ethyleneglycol) and carboxymethyl-chitosan. J. Appl. Polym. Sci. 2013; 127: 308-315.
Sask K N, Berry L R, Chan A K, Brash J L. Modification of polyurethane surface with an antithrombin-heparin complex for blood contact: influence of molecular weight of polyethylene oxide used as a linker/spacer. Langmuir 2011; 28: 2099-2106.
Chung Y C, Choi 1W, Lee J Y, Chun B C. The control of molecular interactions between polyurethane copolymers by grafted malic acid and its impact on polymer characteristics. J. Appl. Polym. Sci. 2012; 126: 225-232.
Wang Y, Xu W, Chen Y. Surface modification on polyurethanes by using bioactive carboxymethylated fungal glucan from Poriacocos. Colloid Surf. B—Biointerfaces. 2010; 81: 629-633.
Lu Y, Shen L, Gong F, Cui J, Rao J, Chen J, Yang W. Polycarbonate urethane films modified by heparin to enhance hemocompatibility and endothelialization. Polym. Int. 2012; 61: 1433-1438.
Tan M, Feng Y, Wang H, Zhang L, Khan M, Guo J, Chen O, Liu J. Immobilized bioactive agents onto polyurethane surface with heparin and phosphorylcholine group. Macromol. Res. 2013; 21: 541-549.
J. Jiang, Y. Fu, Q. Zhang, X. Zhan, F. Chen, Novel amphiphilic poly(dimethylsiloxane) based polyurethanenetworks tethered with carboxybetaine and their combinedantibacterial and anti-adhesive property, Appl. Surf. Sci. 412 (2017) 1-9.
Banoriya D, Purohit R, Dwivedi R K. Advanced application of polymer based biomaterials, Mater. Today. 2017; 4: 3534-3541.
Yuan S, Li Y, Luan S, Shi H, Yan S. Infection-resistant styrenic thermoplastic elastomers that can switch from bactericidal capability to anti-adhesion. J. Mater. Chem. B. 2016; 4: 1081-1089.
Muzzio N E, Pasquale M A, Diamanti E, Gregurec D, Moro M M, Omar A, Sergio E M. Enhanced antiadhesive properties of chitosan/hyaluronic acid polyelectrolyte multilayers driven by thermal annealing: low adherence for mammalian cells and selective decrease in adhesion for Gram-positive bacteria. Mater. Sci. Eng. C. 2017; 80: 677-687.
Craig R G, Power J M. Restorative Dental Materials. 11^{th} edn. St Louis, Mo.: Mosby-Year Book, Inc.; 2002, pp. 614-618.
Osinaga P W, Grande R H, Ballester R Y, Simionato M R, Delgado Rodrigues C R, Muench A. Zinc sulfate addition to glass-ionomer-based cements: influence on physical and antibacterial properties, zinc and fluoride release. Dent. Mater. 2003; 19: 212-217.
Takahashi Y, Imazato S, Kaneshiro A V, Ebisu S, Frencken J E, Tay F R. Antibacterial effects and physical properties of glass-ionomer cements containing chlorhexidine for the ART approach. Dent. Mater. 2006; 22: 467-452.
Yamamoto K, Ohashi S, Aono M, Kokubo T, Yamada I, Yamauchi J. Antibacterial activity of silver ions implanted in SiO2 filler on oral streptococci. Dent. Mater. 1996; 12: 227-229.
Imazato S, Russell R R, McCabe J F. Antibacterial activity of MDPB polymer incorporated in dental resin. J. Dent. 1995; 23: 177-181.
Gottenbos B, van der Mei H C, Klatter F, Nieuwenhuis P, Busscher H J. In vitro and in vivo antimicrobial activity of covalently coupled quaternary ammonium silane coatings on silicone rubber. Biomaterials 2002; 23: 1417-1423.
Murata H. Permanent, non-leaching antibacterial surfaces-2: how high density cationic surfaces kill bacterial cells. Biomaterials 2007; 28: 4870-4879.
Xie D, Weng Y, Guo X, Zhao J, Gregory R L, Zheng C. Preparation and evaluation of a novel glass-ionomer cement with antibacterial functions. Dent. Mater. 2011; 27: 487-496.
Ebi N, Imazato S, Noiri Y, Ebisu S. Inhibitory effects of resin composite containing bactericide-immobilized filler on plaque accumulation. Dent. Mater. 2001; 17: 485-491.
Imazato S, Ebi N, Takahashi Y, Kaneko T, Ebisu S, Russell R R B. Antibacterial activity of bactericide-immobilized filler for resin-based restoratives. Biomaterials 2003; 24: 3605-3609.
Weng Y, Howard L, Chong V J, Guo X, Gregory R L, Xie D. A novel furanone-modified antibacterial dental glass ionomer cement. Acta. Biomater. 2012; 8: 3153-3160.
Weng Y, Howard L, Chong V J, Guo X, Gregory R L, Xie D. A novel antibacterial dental resin composite. J. Mater. Sci. Mater. Med. 2012; 23: 1553-1561.
Xie D, Smyth C A, Eckstein C, Bilbao G, Mays J, Eckhoff D E, Contreras J L. Cytoprotection of PEG-modified adult porcine pancreatic islets for improved xenotransplantation. Biomaterials 2005; 26: 403-412.
Lakshmi S, Kumar S S P, Jayakrishnan A. Bacterial adhesion onto azidated poly(vinyl chloride) surfaces. J. Biomed. Mater. Res. 2002; 61: 26-32.
Xie D. Surface coating for biological implants and prostheses. U.S. Pat. No. 9,550,011 B2, 2017.
Yuan H, Qian B, Chen H, Lan M. The influence of conditioning film on antifouling properties of the polyurethane film modified by chondroitin sulfate in urine. Appl. Sur. Sci. 2017; 426: 587-596.
Kim Y, Farrah S, Baney R H. Membrane damage of bacteria by silanols treatment. Electronic J. Biotechnol. 2007; 10: 252-259.
Guelcher S A, Hollinger J O. An Introduction to Biomaterials. Boca Raton: CRC Press; 2006, pp. 161-183.
Lattmann E, Dunn S, Niamsanit S, Sattayasai N. Synthesis and antibacterial activities of 5-hydroxy-4-amino-2(5H)-furanones. Bioorg. Med. Chem. Lett. 2005; 15: 919-921.
Moffitt E A. Blood substitutes. Canad. Anaesth. Soc. J. 1975; 22: 12-19.
Teodorescu M, Bercea M. Poly(vinylpyrrolidone)—A versatile polymer for biomedical and beyond medical applications. Polym-Plast. Technol. Eng. 2015; 54: 923-943.
Freij-Larsson C, Jannasch P, Wesslen B. Polyurethane surface modified by amphilic polymers: effect on protein adsorption. Biomaterials 2000; 21: 307-315.
Mattson G, Conklin E, Desai S, Nielander G, Savage M D, Morgensen S. A practical approach to crosslinking. Mol. Biol. Rep. 1993; 17: 167-183.
Gibney K, Sovadinova I, Lopez A I, Urban M, Ridgway Z, Caputo G A, Kuroda K. Poly(ethylene imine)s as antimicrobial agents with selective activity. Macromol. Biosci. 2012: 12: 1279-1289.
Shima S, Matsuoka H, Iwamoto T, Sakai H. Antimicrobial action of epsilon-poly-L-lysine. J. Antibiot. 1984; 37: 1449-1455.
Zoumpoulakis P, Camoutsis C, Pairas G, Sokovic M, Glamoclija J, Potamitis C, Pitsas A. Synthesis of novel sulfonamide-1,2,4-triazoles, 1,3,4-thiadiazoles and 1,3,4-oxadiazoles, as potential antibacterial and antifungal agents, biological evaluation and conformational analysis studies. Bioorg. Med. Chem. 2012; 20: 1569-1583.

What is claimed:

1. A material having an antifouling and a biocidal property, the material comprising a polyvinylchloride plastic covalently linked to a polymer, where the polymer comprises an antifouling component and a biocidal component.

2. A material according to claim 1, where the polymer further comprises a coupling component.

3. A material according to claim 2, where the coupling component is N-succinicmidyl acrylate.

4. A material according to claim 1, where the antifouling component of the polymer is N-vinylpyrrolidone.

5. A material according to claim 1, where the biocidal component of the polymer exerts an antibacterial effect.

6. A material according to claim 1, where the biocidal component of the polymer exerts an antibacterial effect against a bacterium selected from the group consisting of P. aeruginosa and S. aureus.

7. A material according to claim 1, where the biocidal component of the polymer comprises 5-acryloylethyleneglycol-3-4-dichloro-2(5H)-furanone.

8. A material according to claim 1, where a molar ratio of the antifouling agent to the biocidal component is from about 87:5 to about 72:20.

9. A material according to claim 1, where a molar ratio of the antifouling component to the biocidal component to the coupling component is from about 87:5:8 to about 72:20:8.

10. A medical device comprising a surface material, where the surface material has an antifouling and a biocidal property, the surface material comprising a polyvinylchloride plastic covalently linked to a polymer, where the polymer comprises an antifouling component and a biocidal component.

11. A medical device according to claim 10, where the polymer further comprises a coupling component.

12. A medical device according to claim 11, where the coupling component is N-succinicmidyl acrylate.

13. A medical device according to claim 10, where the antifouling component of the polymer is N-vinylpyrrolidone.

14. A material according to claim 10, where the biocidal component of the polymer exerts an antibacterial effect.

15. A material according to claim 14, where the biocidal component of the polymer exerts an antibacterial effect against a bacterium selected from the group consisting of P. aeruginosa and S. aureus.

16. A medical device according to claim 10, where the biocidal component of the polymer comprises 5-acryloylethyleneglycol-3-4-dichloro-2(5H)-furanone.

17. A medical device according to claim 10, where a molar ratio of the antifouling agent to the biocidal component is from about 87:5 to about 72:20.

18. A medical device according to claim 10, where a molar ratio of the antifouling component to the biocidal component to the coupling component is from about 87:5:8 to about 72:20:8.

19. A polymer having the structure: 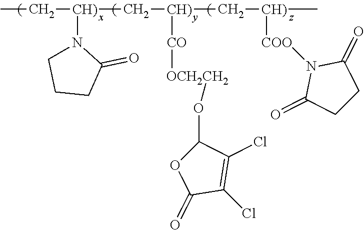
where x, y and z are integers between 1 and 10,000.

20. A polymer according to claim 19, where z=8.

21. A polymer according to claim 19, where the polymer is covalently linked to polyvinylchloride.
